(19) United States
(12) Patent Application Publication
(10) Pub. No.: US 2005/0113930 A1
(43) Pub. Date: May 26, 2005

(54) Method of manufacture, installation, and system for an alveolar ridge augmentation graft

(75) Inventors: Scott D. Ganz, River Vale, NJ (US); Roger C. Stikeleather, Doylestown, PA (US); Thomas J. Bradbury, Yardley, PA (US); Alfred Anthony Litwak, Manasquan, NJ (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Therics, Inc., Princeton, NJ (US)

(21) Appl. No.: 10/789,358

(22) Filed: Feb. 26, 2004

Related U.S. Application Data
(60) Provisional application No. 60/450,407, filed on Feb. 26, 2003.

(51) Int. Cl. A61F 2/28

(52) U.S. Cl. 623/17.17

(57) ABSTRACT

A bone graft that is made at least partially of synthetic material or demineralized bone matrix and is manufactured in suitable shape and/or dimensions to augment an alveolar ridge. The bone graft may be such as to augment both a portion of the crest of the alveolar ridge and a portion of at least one side of the alveolar ridge. The graft may include at least one hole for the intended position of an implant base, and/or at least one hole for attachment hardware. The graft may be manufactured to standard dimensions or it may be manufactured to patient-unique dimensions which may be determined radiographically prior to surgery and prior to manufacturing of the bone graft. The bone graft may be able to be carved for dimensional adjustment during surgery. The bone graft may have composition and/or local geometry which varies from one place to another, and may have a particular composition and/or local geometry at places intended to adjoin natural bone.

### BACKGROUND OF THE INVENTION

1. Field of the Invention

The present invention relates to alveolar ridge augmentation, and more particularly to a bone graft for the alveolar ridge augmentation, a method of manufacturing the bone graft, a method of installing the bone graft and a system for the same.

2. Description of the Related Art

When teeth are missing from either the mandible or the maxilla, resorption of bone usually occurs at the site where the teeth had been. For a variety of purposes, including preparation for an endosseous implant or simply for cosmetic improvement, it may be desirable to augment an alveolar ridge where resorption has occurred. Frequently in such situations, there has been resorption both on the crest of the alveolar ridge and on at least one side of the alveolar ridge.

An endosseous implant (EDI) comprises an implant base that is installed directly into the bone of a patient's mandible or maxilla, and an abutment post that attaches to the implant base, and a tooth prosthesis that attaches to the abutment post. Implant dentistry has become a practical restorative method with a high reliability and success rate. Existing procedures for endosseous implants are described in “An Illustrated Guide to Understanding Dental Implants,” by Scott D. Ganz, D.M.D. (1993). For an EDI to be successful it is necessary that the implant base have an appropriate length which is supported by intimate contact with bone.

In such situations, it has been possible to augment the alveolar ridge and sometimes to install an implant base by adding bony material onto the surface of the alveolar ridge, a procedure called alveolar ridge augmentation.

In some procedures, the alveolar ridge has been augmented using a formable filler material which is intended to become bone or result in the formation of natural bone. For example, the formable material has sometimes been a paste or-putty comprising demineralized bone matrix, bone chips, other components derived from bone, etc. However, such formable material has not always remained where it has been placed and has not always integrated sufficiently well with the existing bone to form a strong foundation for eventual installation of an implant base. Sometimes the formable material has stayed in place and has successfully integrated with existing bone but has later resorbed. This type of procedure has required a time for healing and osseointegration after placement of the graft, before installation of the implant base. With formable material, the installation of the implant base has had to be performed during a later surgery.

Other current procedures for augmenting the alveolar ridge involve installing a bone graft made of allograft or autograft having appropriate shape. Because the autograft or allograft has been a solid material, such a procedure has avoided the migration problem experienced with formable material. However, the bone material installed in such a procedure has still been subject to possible resorption. As is usually the case with such sources of bone material, the use of allograft bone has introduced the possibility of disease transmission from the donor, and the use of autograft bone has involved the extra inconvenience, pain and expense of the surgery at a second site in the same patient for harvesting of bone. Also, the use of such sources of bone material has involved time-consuming carving and shaping of the bone graft during the surgery.

All of the above-mentioned procedures have involved the possibility of resorption of implanted bone material, which would represent a re-occurrence of the original problem.

In regard to surgical technique for use with grafts of any rigid material, both the shaping of the bone graft and the preparation (if done) of the installation site have typically been performed using localized cutting tools such as small burrs. Typically, sequential on-the-spot cutting and fitting have been performed so that the graft fits with the appropriate portion of the alveolar ridge. Typically the graft has had to be inserted, removed, adjusted and re-inserted a number of times during a surgical procedure, with decisions being made as the surgery progressed.

When synthetic materials have been used, current practice has basically only provided uniform composition of material for the bone graft. When synthetic materials have been used they have generally been manufactured of uniform composition, and even if they were manufactured of non-uniform composition, it would not be easy to predict exactly where a particular composition would end up in the bone graft after carving. Providing specific local geometry, such as small channels, on the bone-facing surfaces of a synthetic bone graft has also been nearly impossible. Nevertheless, it is known that osseointegration can be encouraged if the bone-facing surface of a bone graft has particular geometry and/or composition properties.

Accordingly, there remain multiple needs for improvement in procedures for augmentation of the alveolar ridge. It would be desirable to augment, using a single graft, both the crest and at least one side or even both sides of the alveolar ridge. It would be desirable to avoid the problems of migration of formable material. It would be desirable to avoid the problems of second site surgery or possible disease transmission which are inherent with autograft and allograft, respectively. It would be desirable to make the surgical process as efficient as possible by reducing the amount of unrehearsed cutting and fitting which has to take place during surgery. It would be desirable to provide an alveolar ridge augmentation graft which contains one or even more than one pre-manufactured holes suitable for the base of an endosseous implant. It would be desirable to improve the fit between bone graft from any source and the natural bone against which the bone graft is placed, so as to promote integration of natural bone with the bone graft. It would be desirable to provide specific composition and/or local geometry at the bone-facing surfaces of the bone graft. It would be desirable that the graft be able to wick blood and other bodily fluids. It would be desirable to minimize the number of surgical procedures which a patient must undergo.

### BRIEF SUMMARY OF THE INVENTION

An aspect of the invention is a bone graft which is made at least partially of synthetic material or demineralized bone matrix and which is manufactured in suitable shape and/or dimensions to augment an alveolar ridge. The bone graft may be such as to augment both a portion of the crest of the alveolar ridge and a portion of at least one side of the alveolar ridge. The graft may comprise at least one hole for the intended position of an implant base, and/or at least one hole for attachment hardware. The graft may be manufactured to standard dimensions or it may be manufactured to patient-unique dimensions which may be determined radiographically prior to surgery and prior to manufacturing of the bone graft. The bone graft may be able to be carved for dimensional adjustment during surgery. The bone graft may have composition and/or local geometry that varies from one place to another, and may have a particular composition and/or local geometry at places intended to adjoin natural bone.

Other aspects of the invention are a method of manufacture of the bone graft, and methods of installing the bone graft and possibly an implant base. The installation may make use of patient-unique templates for cutting. Another aspect of the invention is a kit comprising the bone graft, tools for its installation, templates and possibly other surgical items.

### BRIEF DESCRIPTION OF THE SEVERAL VIEWS OF THE DRAWINGS

FIG. 1 illustrates the three-dimensional printing process in accordance with the prior art.

FIGS. 2A and 2B are photographs of an exemplary bone grafts in accordance with principles of the present invention.

FIG. 3 illustrates an exemplary bone graft shape having a flat surface suitable to augment a flat crestal surface of an alveolar ridge, and two side regions in accordance with principles of the present invention.

FIG. 4 illustrates an exemplary bone graft shape having a sharp internal crestal corner suitable to augment a sharp crestal surface of an alveolar ridge, and a side surface in accordance with principles of the present invention.

FIG. 5 illustrates an exemplary bone graft shape having a curved internal crestal corner suitable to augment a curved alveolar ridge surface, and a side surface in accordance with principles of the present invention.

FIG. 6 illustrates an exemplary bone graft shape including a completely bounded hole, in this case for an implant base, in accordance with principles of the present invention.

FIG. 7 illustrates an exemplary bone graft shape including a partially bounded hole, in this case for an implant base, in accordance with principles of the present invention.

FIG. 8 illustrates a first step of a surgical procedure to install a bone graft, showing resected gingiva, in accordance with principles of the present invention.

FIG. 9 shows a subsequent step of a surgical procedure to install a bone graft, showing the bone graft in place, in accordance with principles of the present invention.

FIG. 10 illustrates an exemplary bone graft shape including multiple pre-manufactured holes therein in accordance with principles of the present invention.

FIG. 11 illustrates yet another embodiment of the present invention including an exemplary bone graft having multiple pre-manufactured holes therein and further illustrating surface patterns on the graft in accordance with principles of the present invention.

FIG. 12 illustrates an alternative embodiment of the bone graft wherein the graft is composed of a Hydroxyapatite and Tricalcium Phosphate matrix in accordance with principles of the present invention.

### DETAILED DESCRIPTION OF THE INVENTION

Bone Graft

An aspect of the present invention is the bone graft itself. As used herein, the term bone graft is intended to include natural bone (from any source), and processed components of natural bone, and synthetic material of all kinds, and combinations thereof, in a form which is substantially rigid. Some specific types of bone graft are an aspect of the present invention. The bone graft of the present invention may be described both by its geometry and by its material composition.

The bone graft may be made of a substantially rigid material, so that it can have and retain definite shape and dimensions, as opposed to being formable. One possibility is that the bone graft may be manufactured in a non-specific shape intended to be shaped during surgery by removing material from it. Another possibility is that the bone graft may be manufactured to approximate dimensions but may be modified during surgery by removing material from it in local places for dimensional adjustment as required for good fit. Another possibility is that the bone graft may be manufactured to patient-unique dimensions in advance of surgery so exactly that no adjustment or removal of material from it need be made during surgery.

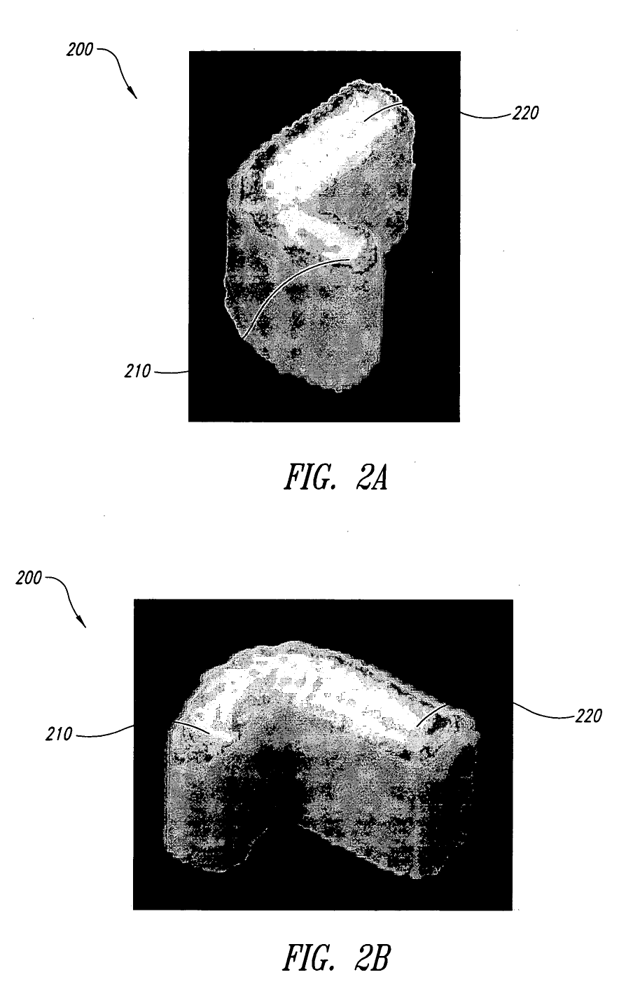

The overall shape of the bone graft may be such as to augment the crest of the alveolar ridge and at least one side of the alveolar ridge. As shown in FIGS. 2A and 2B, the bone graft 200 may have a crestal region 210, which may be shaped and dimensioned to generally fit over a portion of the crest of the alveolar ridge. Connected to the crestal region may be a first side region 220 that may be shaped and dimensioned to generally fit alongside a portion of the alveolar ridge. As shown, the crestal region may be shorter than the first side region and the overall shape may resemble a J, although in general the dimensions of each region depend on a particular patient.

In alternative embodiments, the bone graft 300 of the present invention may comprise a first side region 310, a crestal region 320, and a second side region 330, as shown in FIG. 3. Such a shape may be appropriate when a greater amount of augmentation is desired including augmentation on both sides of the alveolar ridge.

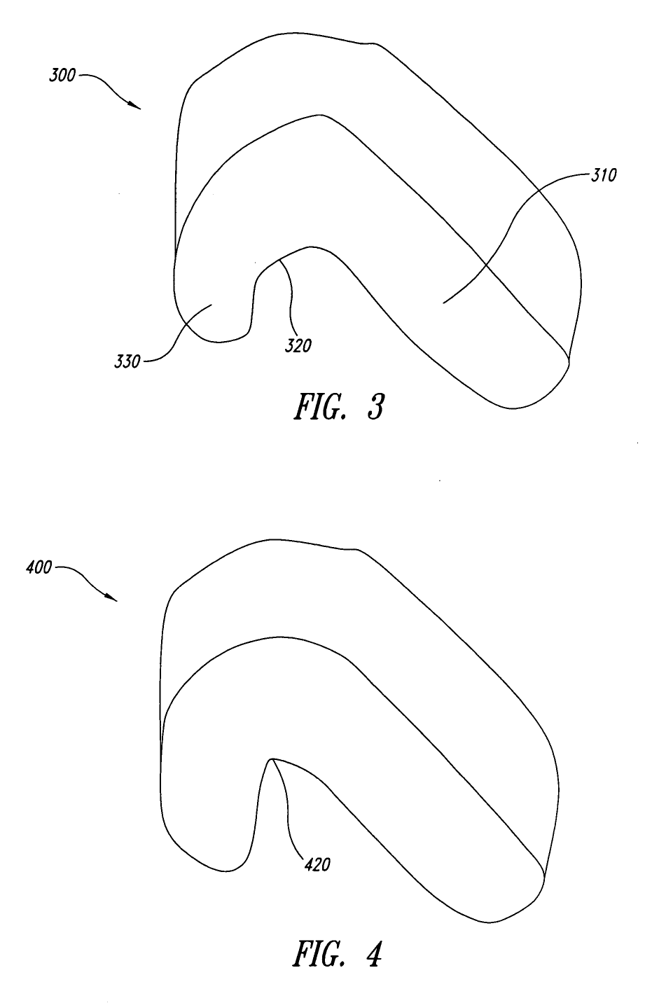

In yet another embodiment, the bone graft 400 may have a shape that has an internal profile or crestal region 420 that is somewhat sharp, and is positioned on its crestal surface that faces the alveolar ridge. This is because, in cases of severe resorption of the alveolar ridge, the alveolar ridge itself may become somewhat sharp. Such a shape is illustrated in FIG. 4.

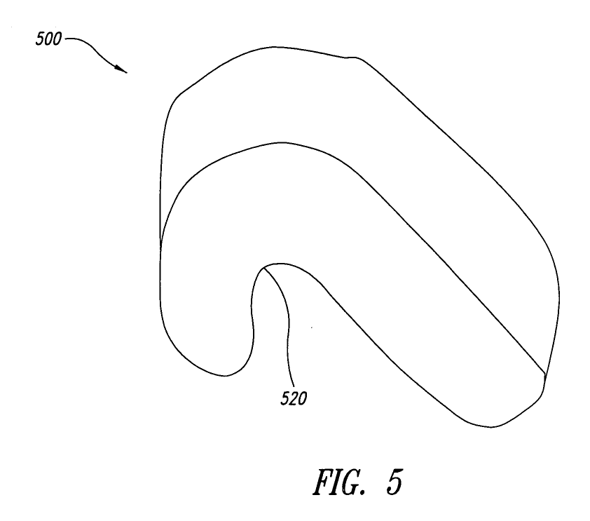

As shown in FIG. 5, yet another embodiment of the present invention is a bone graft 500 having a crestal region 520 that is curved in the region that faces the alveolar ridge.

The illustrative shapes shown in FIGS. 2A, 2B, 3, 4 and 5 are shown without any specific internal architecture such as protrusions, holes, or porosity in them. In terms of dimension of the bone graft along the alveolar ridge, the bone graft may be dimensioned suitably to augment a gap of one or two missing teeth, or more.

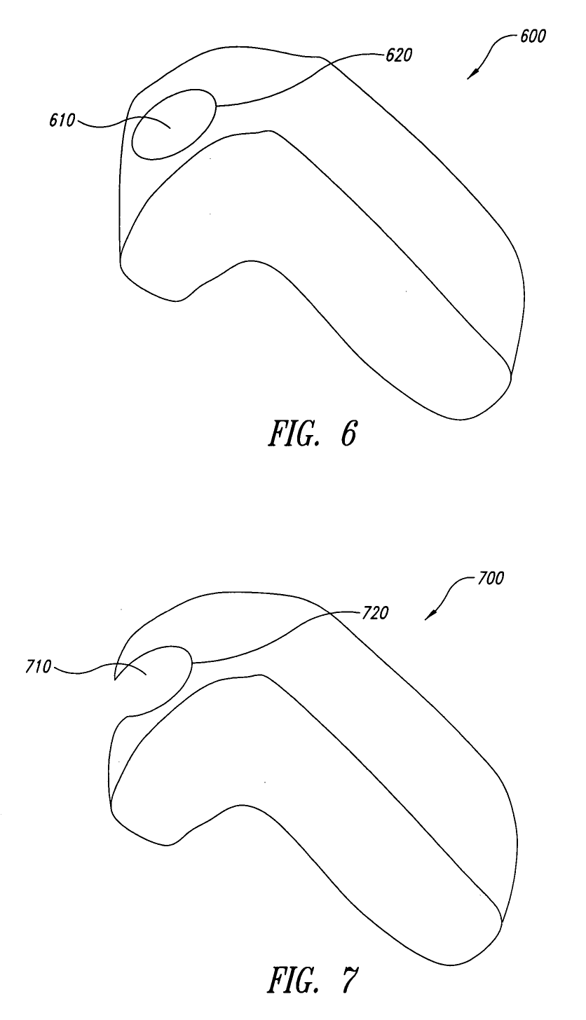

The bone graft may comprise additional geometric features other than its overall shape. The bone graft may comprise one or more features such as an aperture, opening, channel, or hole to assist in the anchoring or installation of one or more implant bases or for any other purpose. As illustrated in FIG. 6, the aperture 610 maybe completely bounded around a top edge 620. In accordance with alternative embodiments of the present invention, the aperture may further be either a blind hole or a through hole extending through the bone graft 600.

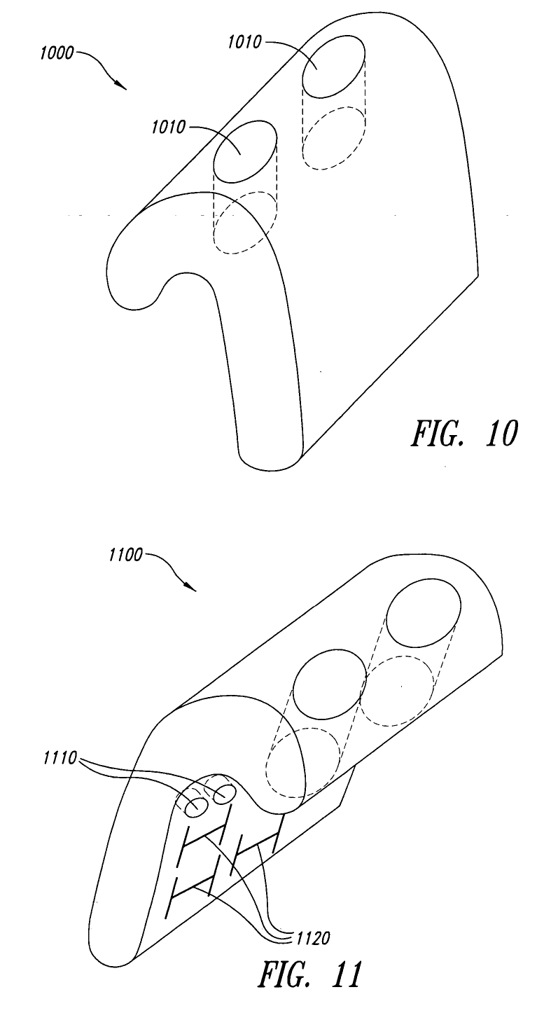

In an alternative embodiment illustrated in FIG. 7, a bone graft 700 may comprise a partially bounded aperture 710 or hole which breaks through an edge of the bone graft along a perimeter 720. This is illustrated in FIG. 7. In further embodiments of the present invention, the bone graft may comprise two or even more apertures or holes, as illustrated in FIG. 10. The opening(s) may be of appropriate dimensions to accommodate the base of an endosseous implant(s). The dimensions, spacing, orientation etc. of the holes may be either standardized or patient-unique. The bone graft may be manufactured with one or more holes for use by a surgical screw or similar attachment device (not shown). Any of the already described shapes could include one or more such holes.

A bone graft for an alveolar ridge augmentation may have a bone-facing surface, which is intended to face natural bone, and a non-bone-facing surface, which is intended not to face natural bone. The non-bone-facing surface of the bone graft may be shaped to provide a desired contour of the gingiva after augmentation. The non-bone-facing surface of the bone graft may be made to be relatively smooth, within the limitations of the manufacturing process described elsewhere herein. For example, the non-bone-facing surfaces may have a surface roughness of less than 300 micrometers r m s. The bone-facing surface of the bone graft may be shaped to have a defined spatial relationship with the shape of the alveolar ridge either as it exists prior to the surgery or as it exists after preparation such as removal of some bone from its surface.

Preparation of the surface of the alveolar ridge (if performed) may be performed with the aid of a template that may be patient-unique. The defined spatial relationship may mean that the bone graft may be closely fitting to the appropriate portion of the alveolar ridge, to within a close tolerance. Alternatively, it may mean that the bone graft could have a predetermined gap, which may everywhere be maintained to within a close tolerance, with respect to the corresponding portion of the alveolar ridge, or the bone graft could have a predetermined amount of interference, which may everywhere be maintained to within a close tolerance, with respect to the corresponding portion of the alveolar ridge.

With the bone graft manufacturing method and the surgical methods described herein, a tolerance of better than 0.4 mm may be achieved on the relative dimensions of the bone graft and the corresponding portion of the alveolar ridge. This tolerance may be applied in the form of either gap or interference as desired, or even a combination of gap in some places and interference in other places.

At a smaller dimensional scale, the bone graft may comprise any of various sorts of channels or similar geometric features which may be conducive to osseointegration. The bone graft may comprise channels within its interior. The bone graft may comprise channels or patterns on at least some exterior surfaces, such as the bone-facing surfaces of the bone graft. For example, such surface patterning may comprise designs such as indented H recesses 1120, grooves, dimples, dead-end holes and any other tire-tread-like designs. Examples are illustrated in FIG. 11 and in co-pending commonly assigned U.S. patent application Ser. No. 60/286,564, herein incorporated in its entirety by reference.

The bone graft may comprise composition which is different at a bone-facing surface as compared to elsewhere in the bone graft, for example, at a soft tissue surface interface. According to aspects of the present invention, the bone graft may have a geometry and/or composition at any surface which is different from its geometry or composition interiorly of the surface. With respect to surface composition, bone-facing surface advantageously is porous and may further include surface geometry in order to enhance bone ingrowth response. One example of a surface geometry is for example, blind holes or dead-end channels 1110. With respect to the tissue-facing surface or interface, the surface is advantageously smooth to promote a healthy soft tissue response which does not include ingrowth. The combination of various aspects of the present invention, including the ability to custom-manufacture a bone graft with prescribed detail, tailored to dimensions of the patient's alveolar ridge, provides confidence that there will not be a need to remove material from, and thereby disturb the pre-designed surface features of, the bone graft at the time of installation in the patient.

The bone graft may comprise a matrix material which exists in the form of particles joined to each other so as to form a three dimensionally interconnected network. In terms of material composition, the matrix material may be or may include a synthetic material. The matrix may be made of a ceramic material which may resemble materials found in natural bone and in particular may be a compound comprising calcium and phosphorus. If the bone graft is made. entirely of synthetic material, that would avoid the possibilities of disease transfer associated with the use of donor bone (allograft) and would avoid the second site surgery associated with autograft.

The matrix material may be nonresorbable. Such a bone graft may be made of or may include nonresorbable hydroxyapatite. The property of nonresorbability may be useful for combating a situation in which natural bone has resorbed. A nonresorbable material that is porous may tend to remain permanently in place while still allowing or encouraging natural bone to grow into its void spaces, thereby resulting in a combination of at least some of the strength of natural bone together with a tendency not to resorb.

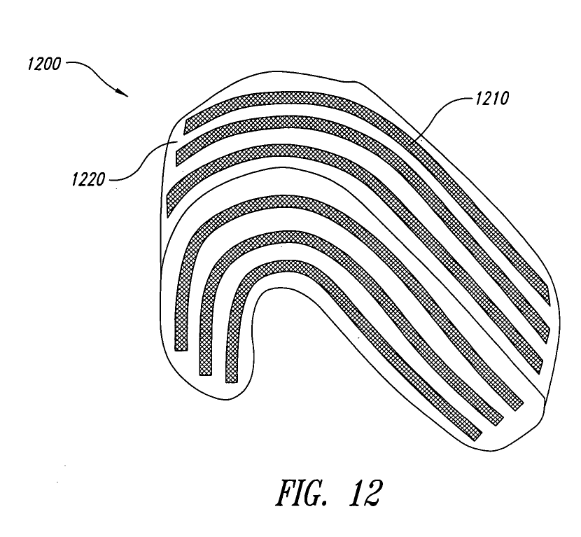

Alternatively, and as shown in FIG. 12, the matrix material may be resorbable or have a resorbable component. According to this embodiment, the resorbable component material may be or may include tricalcium phosphate. It is possible that both nonresorbable 1220 and resorbable materials 1210 may be used in the bone graft 1200. The matrix may contain both hydroxyapatite and tricalcium phosphate, and the proportions of those two substances, or in general any matrix components, may vary from one place to another within the bone graft. The matrix material may be ceramic, as just described or may include other biocompatible materials.

A known problem with autograft augmentation of the alveolar ridge is that the grafted material may resorb, resulting in a reoccurrence of the original problem. Accordingly, it is possible to make the graft with a pattern of hydroxyapatite, which is nonresorbable, while also containing tricalcium phosphate, which is resorbable. The hydroxyapatite may be distributed within the graft in the form of a framework or substantially continuous network of hydroxyapatite which extends to approximately the overall external contours of the graft. The tricalcium phosphate may occupy places not occupied by the hydroxyapatite, while there may also be pores as usual. It is believed that this combination will maintain the overall outline of the graft as implanted, because the hydroxyapatite will generally remain in existence inside the patient's body. However, within the same graft, the tricalcium phosphate will resorb and be replaced by natural bone. It is believed that the natural bone which occupies those places formerly occupied by the tricalcium phosphate will provide a good material structure to support the base of the endosseous implant. For example, the regions of tricalcium phosphate may amount to individual channels within an overall matrix of hydroxyapatite. The cross-sectional dimensions of the channels of tricalcium phosphate may, for example, be in the range from several hundred micrometers to 1 mm.

Alternatively, it is also possible that the matrix material may be or may comprise demineralized bone matrix (DBM), with particles of DBM being joined by a binder substance. Furthermore, the bone graft may comprise polymer particles as the matrix material.

Because the matrix may be porous, it may have pores which may be three dimensionally interconnected. The porosity and the pore size or pore size distribution may be chosen so as to encourage natural bone to grow into the bone graft. The matrix of the bone graft may have pores whose size may be described as a distribution of pore volume as a function of pore size which has a mode at a pore dimension of between 10 micrometers and 25 micrometers. It is also possible that there be at least one other mode in such a pore size distribution. The porosity of the bone graft, which is the fraction of space not occupied by the matrix, may be in the range of from 20% void to 60% void. The pore size and porosity described here, together with the reproducibility of such parameters resulting from the manufacturing process described herein, are such that the material is able to wick blood and other bodily fluids easily and to a great degree. For example, it is believed that the graft of the present invention is capable of wicking up to its own weight in blood or similar aqueous bodily fluids.

The bone graft may further include at least one other material occupying at least some of the pores of the matrix. The bone graft may be osteoconductive or osteoinductive and may comprise additives to give it properties of osteoconduction or osteoinduction, for example, additives which occupy at least some of the pores of the matrix. The bone graft may include demineralized bone matrix (DBM) occupying some of the pores of the matrix. Other possible additive materials can include the patient's own blood products, osteo-active additives, osteogenic additives, growth factors, peptides, bone morphogenic proteins, autogenous growth factors, platelet rich plasma and any of a number of other possible growth-stimulating or biological additives, as described in the patent application referenced below.

If the graft is made with open channels, the open channels may similarly contain demineralized bone matrix or any of the other described additives. It is believed that in order for demineralized bone matrix to be effective in stimulating growth of natural bone, there is an optimum size of the particles of demineralized bone utilized, and the optimum size is at least approximately 200 micrometers. If channels are built into the graft, having channel cross-sectional dimensions in the range of several hundred micrometers to 1 mm, that will allow the channels to be occupied by particles of demineralized bone matrix which are of a size appropriate for stimulating growth of natural bone.

The pores in the matrix of the bone graft may be partially or fully occupied by a polymer, which may be either resorbable or nonresorbable. An example of a resorbable polymer is poly lactic co-glycolic acid (PLGA), and an example of a non-resorbable polymer is poly methyl methacrylate (PMMA). Other examples of each type are given in the patent application referenced below. The polymer may be or may include a comb polymer, as described in U.S. Pat. No. 6,150,459 and elsewhere. The presence of material occupying space in the pores of the matrix may be uniform throughout the bone graft or may be concentrated unequally in certain regions of the bone graft.

The bone graft may be capable of being cut or carved, during surgery or around the time of surgery, to dimensions other than the originally manufactured dimensions, using either powered cutting tools or hand-held cutting tools. The ability to cut or carve the bone graft may be useful for dimensional adjustment and improving fit between the bone graft and the alveolar ridge during surgery, if that becomes necessary.

With regard to its material composition, its design and any other aspects, the bone graft may include any of the features, properties or the like, which are described in co-pending commonly assigned U.S. patent application Ser. No. 60/286,564, which is hereby incorporated by reference.

Method of Installation of Bone Graft

Another aspect of the present invention is a method of installing the bone graft of the present invention.

In preparation for the surgical procedure, the patient may be radiographed and dimensions may be determined of the patient's alveolar ridge. The information may be mathematically represented as a three dimensional solid model. Using that dimensional information, the bone graft may be designed to patient-unique dimensions. The bone graft may be designed fit a portion of the bone structure which was measured in the radiograph. The bone graft may then be manufactured as described elsewhere herein or in co-pending commonly assigned U.S. patent application Ser. No. 60/286,564. Alternatively, it is possible to use a standard or approximate size and shape of a bone graft, which may be manufactured as described elsewhere herein, and to shape it as desired during the surgery. It is also possible to use a nonspecific shape such as a block, which may be manufactured as described elsewhere herein, and to shape it as desired during the surgery.

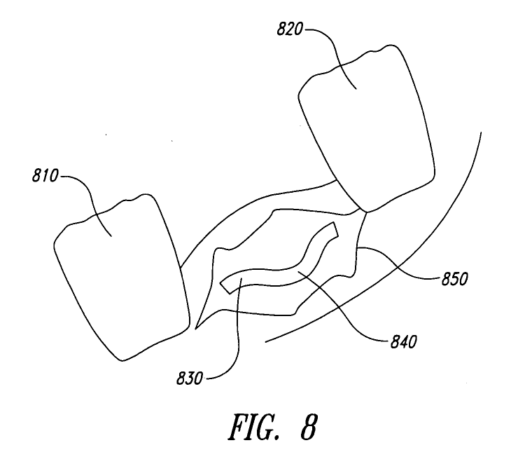

As illustrated in FIG. 8, during the surgical procedure of the present invention, the gingival 850 may be resected. Next, preparation may be done of the site where the bone graft is to be placed by removing only soft tissue, or by additionally removing some bone. In some instances, such as if the bone graft is manufactured to fit with radiographically determined bone dimensions, it may be desired to remove only soft tissue and leave the underlying bony material substantially undisturbed. For removal of soft tissue, it is possible to use none-bone-cutting tools, either hand-held or powered, which are capable of cutting soft tissue but are not sufficiently hard to cut bone. In other instances, it may be desired to also remove some of the bony material, in addition to soft tissue. For this, bone-cutting tools may be used. Templates, which may be patient-unique, may be used to guide the cutting.

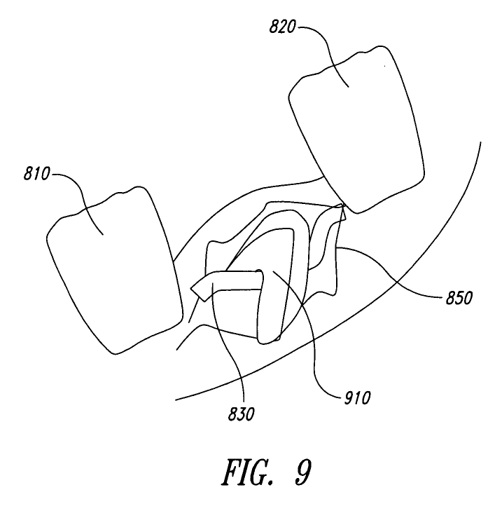

As shown in FIG. 8, between existing teeth 810, 820, the gingival 850 is resected to expose the crest of the alveolar ridge 830. In this illustration, the alveolar ridge crest dips 840 at the location of the missing tooth. After the alveolar ridge and/or other bone has been exposed and possibly prepared as described above, the bone graft of the present invention may be brought into contact with the alveolar ridge. The positioning of the graft 910 is illustrated in FIG. 9. The fit between the bone graft and the alveolar ridge may be such that no dimensional adjustment is needed during surgery. Alternatively, some adjustment to improve fit may have to be done during the surgical procedure, such as by removing material from the bone graft in selected locations or removing material from the alveolar ridge or both.

It may be necessary to repeatedly bring the bone graft into contact with the alveolar ridge, check fit, remove the bone graft and adjust either the bone graft or the alveolar ridge. Alternatively, the geometry of the alveolar ridge and the design of the bone graft may be such that after the bone graft has been brought into the alveolar ridge, the bone graft may be maintained in sufficient contact with adjacent natural bone simply by virtue of its shape and dimensions.

Alternatively, the bone graft may require some anchoring in order to maintain contact with the adjacent natural bone. If such anchoring is needed, appropriate anchoring may be performed at this point during the surgical procedure, such as installation of surgical screws. If this is planned, appropriate features such as holes may be provided in the design of the bone graft to accommodate such anchoring, as further illustrated by the bone grafts shown in FIGS. 10 and 11.

An implant base for an endosseous implant may be installed in both the bone graft and the underlying bone during the same surgical procedure in which the bone graft itself is installed in the patient. In this event, the implant base may also serve as anchoring to keep the bone graft in place. The bone graft may be manufactured with appropriate holes or other features for the implant bases. Preparation of the implant base site may be performed using a template which may be patient-unique such as to locate and/or orient the drills which drill holes for the implant base. However, it is not required that the implant base be installed during the same surgical procedure as the bone graft. It is possible that installation of the implant base may be done separately in a later procedure after there has been some amount of healing and integration of the bone graft with natural bone. In alternative embodiments, the bone graft is pre-manufactured with multiple holes for as many corresponding implant bases as may be desired.

During the later stages of the surgical procedure, appropriate surgical substances may be applied as appropriate. Antiseptic and/or antibiotic may be applied before the bone graft is put into place permanently. Even though the bone graft may be a close fit with the adjacent bone, it is possible that formable material such as putty containing bone-derived substances may be applied for filling possible gaps between the bone graft and the adjacent bone or simply to improve the interaction between the bone graft and the adjacent bone. A surgical membrane such as Gore-Tex or collagen may be used to inhibit the growth of soft tissue in certain places. The gingiva may then be closed. Suturing may be performed. If installation of the implant base is performed during the surgical procedure, it is also possible that a temporary abutment post or even an abutment post plus a tooth prosthesis may be installed onto the implant base during the same surgical procedure.

Use of Templates

Another aspect of the method of the present invention is the use of templates. If it is planned to install implant base(s) during the same surgical procedure as the alveolar ridge augmentation, the installation of the implant base(s) may be aided by an implant base template which is suitable to guide drills or other tools for installation of an implant base. The implant base template may be derived at least in part from patient-unique data which may be radiographic data. The implant base template may take its overall location from one or more teeth or other features in the patient's mouth. The implant base template may, for example, be made of polymer by stereolithography using the same set of solid modeling data used for other aspects of surgical planning, and may comprise drill bushings to locate and orient drills. If more than one drill is used in succession to prepare the site for the implant base, more than one implant base template may be created and used.

It is also possible to use templates, which may be patient-unique, during the cutting of bone or other tissue for preparation of the site before the bone graft is brought in.

Method of Manufacture of Bone Graft

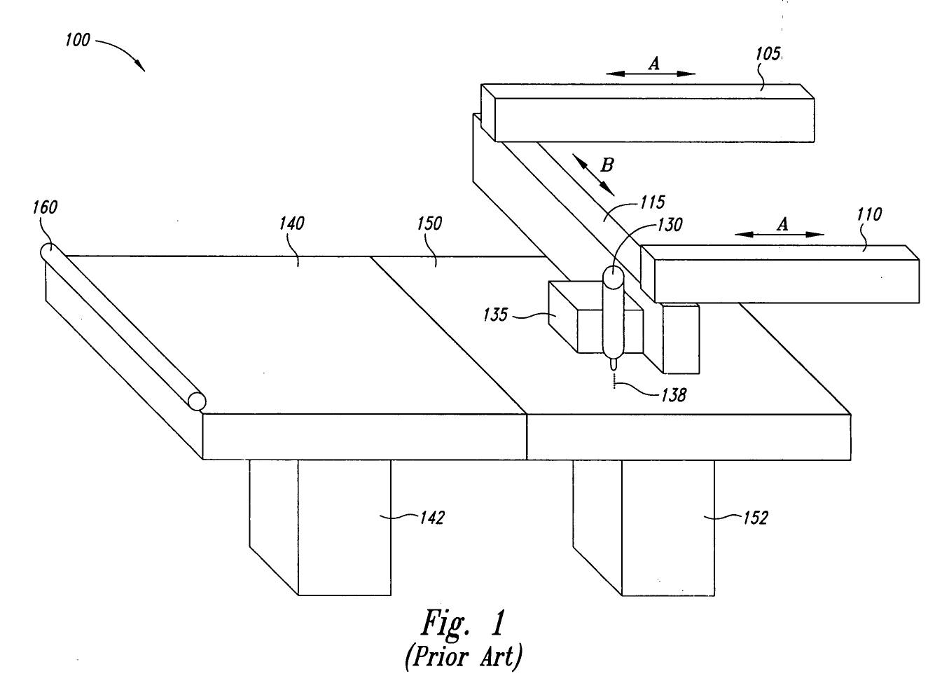

The bone graft of the present invention may be manufactured by methods that include three-dimensional printing (3DP). Three-dimensional printing process described in U.S. Pat. No. 5,204,055 and elsewhere, is the manufacture of objects by assembling them from powder in a layer-by-layer fashion. FIG. 1 illustrates one exemplary three-dimensional printing apparatus 100 in accordance with the prior art. The apparatus 100 includes a roller 160 for rolling powder from a feed bed 140 onto a build bed 150. Vertical positioners, 142 and 152 position the feed bed 140 and the build bed 150 respectively. Slow axis rails 105, 110 provide support for a printhead 130 in the direction of slow axis motion A, and fast axis rail 115 provides support for the printhead 130 in the direction of fast axis motion B. The printhead 130 is mounted on support 135, and dispenses liquid binder 138 onto the build bed 150 to form the three-dimensional object.

In accordance with the three-dimensional printing (3DP) process, layers of powder can be deposited by roller-spreading or by other means. In selected places powder particles are joined to other powder particles and to other bound regions by the action of a binder liquid which may be dispensed from a dispenser which may resemble an ink-jet printer. Binding can occur as a result of a non-volatile substance being deposited by the binder fluid or dissolved by the binder fluid as the binder fluid lands on the powder bed, or can occur as a result of dissolution of powder particles followed by re-solidification. Unbound powder supports bound regions during printing and can later be removed after completion of 3DP. If appropriate geometric description is available and appropriate software instructions for 3DP can be generated, geometrically complicated articles can be made essentially just as easily as geometrically simple articles can be made.

In the case of articles made with discrete regions of hydroxyapatite in some places and discrete regions of tricalcium phosphate in other places, such articles can be made by the chemical conversion between hydroxyapatite and tricalcium phosphate as a result of reactant deposited in prescribed places during three dimensional printing, as described in co-pending commonly assigned U.S. patent application Ser. No. 60/286,564, herein incorporated in its entirety by reference.

Implantable bone substitutes can be made by using powder which is a ceramic substance which may resemble substances found in natural bone. Possible substances include hydroxyapatite, tricalcium phosphate and other calcium-phosphorus compounds. Powders of different compositions can be deposited in a spatially non-uniform pattern by methods described in the reference below. Such articles may involve a sintering step after the completion of 3DP.

The sintering may be partial sintering, which may be carried out at a combination of temperature and time such that the powder particles partially join directly to each other and yet leave some porosity between them. During the heating leading up to partial sintering, the binder substance may exit from the article in the form of vapor or gaseous decomposition products. During partial sintering the powder particles themselves may soften so as to partially join each other, while still leaving a controlled amount of porosity between them. If a ceramic-sintering step is used, it is likely to be the highest-temperature step in the entire manufacturing sequence, and to be a step which is incompatible with organic substances. If such organic substances are desired in a sintered ceramic bone graft, they may be added after completion of a sintering step.

Implantable bone substitutes can also be made of or can contain non-ceramic substances including demineralized bone matrix (DBM) and polymers. It is possible that the bone graft may be made by spreading powder which is or comprises demineralized bone matrix (DBM), i.e., DBM would be the matrix material, and joining those powder particles to each other using a binder substance. Because of the temperature limitations of DBM, the manufacture of such an article would not involve sintering at elevated temperature after 3DP. It is similarly possible that the article could be manufactured by spreading powder particles of polymer and joining them to each other either by dissolution/resolidification or by a binder substance. Again, there would be temperature limitations much lower than the temperatures used in sintering ceramics.

Addition of biological substances, polymers or other temperature-sensitive substances to the bone graft may be performed after the sintering step if a sintering step is used, or after the basic 3DP-manufacturing step. Such addition of biological substances may be performed, for example, by dipping the bone graft into a liquid solution or by infusing liquid into some or all of the bone graft. In the case of polymers, the polymer may be dissolved in a solvent such as chloroform, which may then be allowed to evaporate. The additive(s) can be deposited in a spatially non-uniform pattern by methods described in the reference below. Demineralized bone matrix is one substance which can be added as a later step. Specifically, if after the sintering step the graft comprises empty channels of cross-sectional dimension at least approximately 200 micrometers, interior cavities or compartments, at least some of those channels or cavities may be filled with a flowable substance comprising demineralized bone matrix. Whatever substance is flowed in, in addition to the actual demineralized bone matrix particles themselves, can either be left as is or can be allowed to dry out or evaporate.

These techniques and others are further described in co-pending commonly assigned U.S. patent application Ser. No. 60/286,564. In regard to designing the bone graft uniquely for a particular patient, such as from radiographic data, appropriate techniques are described in co-pending commonly assigned U.S. patent applications Ser. No. 09/828,504 and Ser. No. 09/972,832. The techniques described therein can also be used for designing templates for use during the surgical procedure, and for manufacturing (if desired) a physical model of an appropriate portion of the patient's skull for surgical planning purposes.

Kit

Another aspect of the present invention is a kit comprising components which may be used during the described surgical procedure.

The kit may comprise at least one bone graft of the present invention intended for implantation in the patient. The dimensions of the bone graft(s) may be coordinated with any or all of: appropriate dimensions of the alveolar ridge including the extent of bone resorption/degradation in the patient; and dimensions and intended position of an implant base intended to be installed in the alveolar ridge and the bone graft. In addition to a first bone graft intended for implantation into the patient, the kit may further include a duplicate bone graft for use in case of unexpected need during surgery, and/or may include a bone graft which is oversized such that it could be carved or fitted to size during surgery if needed, and/or may even include a bone graft which is a featureless block of material, any of which could be cut to fit during surgery if needed. Alternatively, the kit may simply comprise bone grafts having standard dimensions or a variety of standard dimensions.

The kit may also comprise one or more cutting tools such as for preparing the alveolar ridge. The kit may comprise bone-cutting tools which are suitable for cutting bone. The kit may comprise non-bone-cutting tools, either hand-held or powered, which are suitable for cutting soft tissue but not suitable for cutting bone. If the implant base is planned to be installed during the same surgery as the bone graft, the kit may also comprise at least one implant base template for determining the position and direction of drilling for installation of implant bases. For installation of implant bases, the kit may further include the implant base(s), and implant base site preparation tools such as drills, and an implant base installation tool.

The kit may further include a handling tool for placing the bone graft onto the alveolar ridge. The kit may include a surgical membrane such as GoreTex or collagen suitable to block the growth of soft tissue in desired places. The kit may include surgical screws or similar hardware suitable for attaching the bone graft, and tools suitable for installing the surgical screws. The kit may include antiseptics and/or antibiotics. The kit may further include formable filler materials suitable for filling possible gaps between the bone graft and adjacent bone, or, alternatively, for use as the entire filler material. The kit may include suture materials. The kit may be designed so that it, or appropriate components of it, are sterilized and packaged or otherwise maintained in a sterile condition.

Further Comments

It can be appreciated that the bone graft of the present invention comprises a synthetic material conducive to the ingrowth of natural bone which has not heretofore been available in customized shapes for use in alveolar ridge augmentations. The described bone graft is a rigid article which may be made partially or entirely of synthetic material or demineralized bone matrix, and is conducive to the ingrbwth of natural bone. The bone graft will not migrate. If the bone graft comprises hydroxyapatite, the hydroxyapatite itself does not resorb, meaning that the bone graft will not completely disappear. The bone graft can include an extent of designed detail, as far as composition, surface texturing and/or geometry (including local geometry), which has not heretofore been available. The bone graft of the present invention can provide a degree of dimensional matching to the patient's alveolar ridge, at the time of manufacture of the bone graft, which has not heretofore been available.

It can also be appreciated that the described bone graft and procedure greatly increase the amount of planning and dimensional determination that can be done in advance of surgery, thereby reducing some of the work which normally has to be done during surgery. The bone graft can be manufactured ahead of time to exact patient-unique dimensions. This can potentially improve the quality of fit between the bone graft and the alveolar ridge, perhaps approaching the quality of fit of a filler made entirely of formable material, which should be conducive to bone ingrowth. At the same time, the amount of surgical time and labor required for installing such a bone graft would be relatively small, roughly comparable to the surgical time for installing a formable filler material. This would be achieved without much repetitive cutting and fitting during surgery, and so should not require a long surgical procedure.

It can also be appreciated that the simultaneous use of multiple aspects of the present invention provides abilities not heretofore available. The custom dimensioning and custom manufacture of the bone graft may provide the ability to create a desired fit during surgery with little or no unrehearsed cutting-to-fit or adjustment during the surgical procedure. It becomes possible to design and manufacture a bone graft of precise dimension which has a specified geometry and/or composition at those surfaces which are intended to abut the natural bone such as the alveolar ridge, and which has some other different specified geometry or composition internally, and to be confident that the alveolar ridge will match closely with the pre-manufactured surface of the bone graft and that there will not be a need to remove material from the surface of the bone graft (which might alter the designed surface-unique geometry or composition) for purposes of fitting.

All patents and applications cited above are incorporated by reference in their entirety. Furthermore, Provisional Patent Application No. 60/450,411 entitled Method and System for Repairing Endosseous Implants, Such as With a Bone Graft Implant, filed Feb. 26, 2003, and the non-provisional application claiming priority to the same; Provisional Patent Application No. 60/450,410 entitled Method of Manufacture, Installation, and System for a Sinus Lift Bone Graft, filed Feb. 26, 2003, and the non-provisional application claiming priority to the same are hereby incorporated in their entirety by reference.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. These and other changes can be made to the invention in light of the above detailed description.

From the foregoing it will be appreciated that, although specific embodiments of the invention have been described herein for purposes of illustration, various modifications may be made without deviating from the spirit and scope of the invention. In general, in the following claims, the terms used should not be construed to limit the invention to the specific embodiments disclosed in the specification and the claims, but should be construed to include all methods, apparatus and articles that operate under the claims. Accordingly, the invention is not limited by the disclosure, but instead the scope of the invention is to be determined entirely by the following claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A bone graft suitable to augment an alveolar ridge, wherein the bone graft comprises synthetic material or demineralized bone matrix in a rigid form manufactured to approximately mate with a void on the alveolar.

2. The bone graft of claim 1, wherein the bone graft comprises a crestal region suitable to augment a crest of the alveolar ridge and a first side region suitable to augment a first side of the alveolar ridge.

3. The bone graft of claim 2, wherein the crestal region has a flat portion facing the alveolar ridge.

4. The bone graft of claim 2, wherein the crestal region has a curved portion facing the alveolar ridge.

5. The bone graft of claim 2, wherein the crestal region has an internally angled portion facing the alveolar ridge.

6. The bone graft of claim 2, wherein the bone graft further comprises a second side region suitable to augment a second side of the alveolar ridge.

7. The bone graft of claim 1, wherein the bone graft is suitable to augment the alveolar ridge space represented by one or two missing teeth.

8. The bone graft of claim 1, where the shape or dimensions of the bone graft are chosen based on dimensions of bone in a particular patient.

9. The bone graft of claim 1, wherein the shape or dimensions of the bone graft are chosen based on radiographic data from a particular patient.

10. The bone graft of claim 1, wherein the bone graft has external surfaces facing adjacent natural bone in a patient, which coincide with the adjacent natural bone to within a tolerance of less than 0.4 mm.

11. The bone graft of claim 1, wherein the bone graft comprises non-bone-facing surfaces suitable to provide a desired contour of gingiva after augmentation.

12. The bone graft of claim 1, wherein the bone graft comprises non-bone-facing surfaces having a porosity less than the porosity of a bone-facing surface on the bone graft.

13. The bone graft of claim 1, wherein the bone graft comprises at least one hole corresponding to an intended position of an implant base.

14. The bone graft of claim 1, wherein the bone graft comprises at least one hole corresponding to an intended position of an attachment device.

15. The bone graft of claim 1, the bone graft comprises a matrix of particles joined to each other forming a three dimensionally interconnected network, the matrix has pores wherein the distribution of pore volume as a function of pore size has a mode between 10 micrometers and 25 micrometers.

16. The bone graft of claim 14, wherein the matrix has a porosity between approximately 0.2 and approximately 0.6.

17. The bone graft of claim 1, wherein the bone graft comprises a ceramic.

18. The bone graft of claim 1, wherein the bone graft comprises at least one substance selected from the group consisting of hydroxyapatite, tricalcium phosphate and other calcium-phosphorus compounds.

19. The bone graft of claim 1, wherein the bone graft comprises nonresorbable material.

20. The bone graft of claim 1, wherein the bone graft comprises resorbable material.

21. The bone graft of claim 1, wherein the bone graft comprises both nonresorbable and resorbable materials.

22. The bone graft of claim 1, wherein the bone graft comprises both nonresorbable and resorbable materials in varying proportions in preselected places within the bone graft.

23. The bone graft of claim 1, further comprising channels which extend into an interior.

24. The bone graft of claim 1, further comprising channels or patterns on a surface.

25. The bone graft of claim 1, wherein the bone graft comprises a bone-facing surface having a surface geometry which is different from a geometry at another place in the bone graft.

26. The bone graft of claim 1, wherein the bone graft comprises a bone-facing surface having a surface composition which is different from a composition at another place in the bone graft.

27. The bone graft of claim 1, wherein the bone graft comprises a bone-facing surface having a surface geometry suitable to promote osseointegration.

28. The bone graft of claim 1, wherein the bone graft comprises a bone-facing surface having a surface composition suitable to promote osseointegration.

29. The bone graft of claim 1, further comprising osteoconductive or osteoinductive substances.

30. The bone graft of claim 1, further comprising substances from a patient's own blood, other biological substances or demineralized bone matrix, osteo-active additives, osteogenic additives, growth factors, peptides, bone morphogenic proteins, autogenous growth factors, or platelet rich plasma.

31. The bone graft of claim 1, further comprising a polymer.

32. The bone graft of claim 31, wherein the polymer is a comb polymer.

33. The bone graft of claim 31, wherein the polymer is resorbable.

34. The bone graft of claim 31, wherein the polymer is non-resorbable.

35. The bone graft of claim 1, wherein the bone graft is sterile.

36. The bone graft of claim 1, wherein the bone graft is manufactured at least in part by three dimensional printing.

37. A bone graft suitable to augment an alveolar ridge, wherein the bone graft comprises synthetic material or demineralized bone matrix in a rigid form and has a composition which is different from one place to another within the bone graft.

38. The bone graft of claim 37, wherein the bone graft comprises particles joined together to form a three dimensionally interconnected matrix, and the composition of the matrix is different from one place to another within the bone graft.

39. The bone graft of claim 37, wherein the bone graft matrix further includes additives, wherein a composition of the additives varies from one place to another within the bone graft.

40. A method of installing a bone graft to augment an alveolar ridge, comprising:
manufacturing to specific dimensions and/or shape a bone graft comprising a rigid synthetic material or demineralized bone matrix the bone graft including predetermined apertures for anchoring the bone graft to the ridge;
resecting gingiva; and
installing, in contact with the alveolar ridge, the bone graft.

41. The method of claim 40, wherein the bone graft has at least one dimension which is selected based on characteristics of a particular site in a particular patient.

42. The method of claim 40, wherein at least one dimension of the bone graft is determined using radiographic data.

43. The method of claim 40, wherein the bone graft has at least one dimension which is selected based on a dimension of the implant base.

44. The method of claim 40, wherein installing the bone graft comprises cutting using a bone-cutting tool.

45. The method of claim 40, wherein installing the bone graft comprises cutting using a non-bone-cutting tool which is capable of cutting soft tissue but incapable of cutting bone.

46. The method of claim 40, further comprising, before installing the bone graft, applying a formable filler material between the bone graft and the alveolar ridge.

47. The method of claim 40, wherein installing the bone graft comprises attaching the bone graft.

48. The method of claim 40, wherein installing the bone graft comprises installing an implant base through the bone graft and into the alveolar ridge.

49. The method of claim 48, wherein installing the implant base comprises using an implant base template which is unique to a particular patient.

50. The method of claim 49, wherein at least one dimension of the implant base template is determined using radiographic data.

51. The method of claim 40, further comprising, before installing the bone graft, applying antiseptic and/or antibiotic.

52. The method of claim 40, further comprising, after installing the bone graft, applying a surgical membrane.

53. The method of claim 40, further comprising, after all of the other steps, putting the resected gingiva back in place.

54. A method of installing a bone graft to augment an alveolar ridge, comprising: resecting gingiva; and installing, adjacent to the alveolar ridge, a rigid bone graft; and installing an implant base in the bone graft and the alveolar ridge using an implant base template which is unique to a particular patient.

55. The method of claim 54, wherein at least one dimension of the implant base template is determined using radiographic data.

56. A method of manufacturing a bone graft for augmenting an alveolar ridge, comprising:
depositing successive layers of a powder; and
printing a binder to form a three dimensional article suitable to at least approximately fit the dimensions of a resorbed or missing portion of the alveolar ridge.

57. The method of claim 56, wherein the powder comprises demineralized bone matrix.

58. The method of claim 56, wherein the powder comprises ceramic.

59. The method of claim 58, wherein the depositing of the powder is performed so that at least one characteristic of the powder differs from one place to another within the bone graft.

60. The method of claim 58, further comprising, after the three dimensional printing, heating the article sufficiently to partially sinter the powder together.

61. The method of claim 56, further comprising, after all the described steps, introducing an additional substance into pores of the bone graft.

62. The method of claim 61, wherein the additional substance comprises substances from a patient's own blood, other biological substances or demineralized bone matrix, osteo-active additives, osteogenic additives, growth factors, peptides, bone morphogenic proteins, autogenous growth factors, or platelet rich plasma.

63. The method of claim 61, wherein the additional substance is deposited so as to have a nonuniform composition or concentration from one place to another in the bone graft.

64. An article manufactured by the method of claim 56.

65. A kit for installing a bone graft in an alveolar ridge, comprising: at least one bone graft having bone graft dimensions which are unique to a particular patient, and at least one cutting tool.

66. The kit of claim 65, wherein at least one bone graft dimension is coordinated with dimensions of the alveolar ridge.

67. The kit of claim 65, further comprising a patient-unique implant base template suitable to guide an installing of an implant base.

68. The kit of claim 65, wherein the bone graft comprises synthetic material.

69. The kit of claim 65, wherein the kit comprises at leas t one tool capable of cutting bone and at least one tool which is incapable of cutting bone.

70. The kit of claim 65, further comprising at least one additional article selected from the group consisting of: a carrier for gripping the bone graft, templates, surgical screws, tools for installing surgical screws, formable filler material, antiseptics, antibiotics, a surgical membrane, and sutures.

71. The kit of claim 65, wherein at least some articles in the kit are sterile.
